(19) United States
(12) Patent Application Publication
(10) Pub. No.: US 2011/0238069 A1
(43) Pub. Date: Sep. 29, 2011

(54) THREADED HOLE FORMING DEVICE

(75) Inventors: Eric Zajac, Naples, FL (US); Robert Sluss, Naples, FL (US); James Bradley, Pittsburg, PA (US)

(73) Assignee: Arthrex, Inc., Naples, FL (US)

(21) Appl. No.: 13/025,765

(22) Filed: Feb. 11, 2011

Related U.S. Application Data
(60) Provisional application No. 61/303,546, filed on Feb. 11, 2010.

(51) Int. Cl. A61B 17/16

(52) U.S. Cl. 606/79

(57) ABSTRACT

A hole forming device useful for creating holes with threads in bone is disclosed. The hole forming device may be formed from an inner shaft and outer sleeve extending distally from a handle. The inner shaft may include a pointed tip that may be used to initiate hole formation. Once the hole forming device has been partially inserted into a material, such as, but not limited, bone, the inner shaft may be removed without having to remove the outer sleeve. The outer sleeve remains in place in the material and may then be advanced to create threads within the hole. Screws and other appropriate devices may be secured in the threaded hole.

### CROSS-REFERENCE TO RELATED APPLICATION

This patent application claims the benefit of U.S. Provisional Pat. App. No. 61/303,546, filed Feb. 11, 2010, which is incorporated by reference in its entirety.

### FIELD OF THE INVENTION

The invention relates to hole forming devices, and more particularly, to threaded hole forming devices.

### BACKGROUND

Orthopedic surgeons have used punches to initiate hole formation within bone in a patient. The holes are typically formed by placing a pointed punch in contact with the surface of a bone in a patient. The opposite end of the punch is then struck repeatedly with a hammer to initiate hole formation. The punch is then removed and either a threaded tap or an orthopedic screw is inserted into the hole. The threaded tap cuts into the bone. Inserting the threaded tap if often fraught with challenges. Specifically, it is often challenging to get the end of the first thread cuts into the bone. Many times, multiple attempts must be made before the first thread cuts into the bone, thereby creating needless time delay.

### SUMMARY OF THE INVENTION

This invention is directed to a hole forming device useful for creating holes with threads in bone or other appropriate materials. The hole forming device may be formed from an inner shaft and outer sleeve extending distally from a handle. The inner shaft may include a pointed tip that may be used to initiate hole formation. Once the hole forming device has been partially inserted into a material, such as, but not limited, bone, the inner shaft may be removed without having to remove the outer sleeve. Thus, the inner shaft facilitates insertion of the outer sleeve into the material where the threads on the outer sleeve may engage the bone. The outer sleeve may then be advanced to create threads within the hole. Screws and other appropriate devices may be secured in the threaded hole after the outer sleeve has been removed.

The hole forming device may be formed from a handle and an outer sleeve supported by the handle. The outer sleeve may extend distally from the handle. The outer sleeve may include a hollow chamber extending an entire length of the outer sleeve with a proximal opening in the handle and a distal opening. The hole forming device may include an inner shaft positioned within the outer sleeve and including a pointed tip that extends distally from the distal opening of the outer sleeve. The outer sleeve may include at least one thread extending radially outward from an outer surface of the outer sleeve near the distal end of the outer sleeve. A proximal end of the inner shaft may be exposed such that a proximal end of the inner shaft can be struck while the inner shaft is positioned in the outer sleeve. The outer sleeve may be a generally elongated hollow sleeve with a cylindrical outer surface and a tapered outer surface at the distal end of the outer sleeve facilitating the outer sleeve being driven into bone.

The inner shaft may also include a head coupled to the inner shaft at the proximal end. The handle may include a head cavity positioned at a proximal end of the handle such that at least a portion of the head on the inner shaft resides within the head cavity. The inner shaft may have an outer diameter that provides a smooth transition to the outer sleeve at a location on the inner shaft that coincides with a location of a distal end of the outer sleeve when the inner shaft is positioned in the outer sleeve. An overall length of the inner shaft may be greater than a combined overall length of an assembled outer sleeve and handle. The inner shaft may be a cannula such that the inner shaft is hollow. In another embodiment, the inner shaft may also be formed from a solid material.

The hole forming device may include a releasable connector that releasably attaches the inner shaft to the outer sleeve. In one embodiment, the releasable connector may be formed from at least one thread that extends radially outward from an outer surface of the inner shaft and at least one thread extending radially inward from an inner surface forming the hollow chamber in the outer sleeve such that the at least one thread of the inner shaft and the outer sleeve may interlock. The at least one thread that extends radially outward from the outer surface of the inner shaft may be positioned near the proximal end of the inner shaft, and the at least one thread that extends radially inward from the inner surface of the outer sleeve may be positioned near the proximal end of the handle. The handle may have a generally cylindrical outer surface or other appropriate configuration.

During use, the hole forming device may be placed in contact with a material into which a hole is desired to be created. A hammer, or other appropriate force delivering device, may be used to hit the head of the inner shaft contained within the handle and the outer sleeve while the hole forming device is held in place by grasping the handle. Once the pointed tip has pierced the material and a distal end of the outer sleeve contacts the material, the inner shaft may be removed entirely from the handle or backed out enough to draw the pointed tip of the inner shaft into the outer sleeve while keeping the outer sleeve in place in the material. The handle may then be rotated such that the threads on the outer sleeve cut into the material forming the wall of the hole. The threads on the outer sleeve cause threads to be formed in the wall forming the hole, thereby creating a threaded hole once the outer sleeve is removed from the hole.

These and other embodiments are described in more detail below.

### BRIEF DESCRIPTION OF THE FIGURES

FIG. 1 is a perspective view of a hole forming device.

FIG. 2 is another perspective view of the hole forming device shown in FIG. 1.

FIG. 3 is a cross-sectional view taken at section line 3-3 in FIG. 2.

FIG. 4 is a partial cross-sectional view of an alternative embodiment of the hole forming device, wherein the cross-section is taken at section line 4-4 in FIG. 2.

### DETAILED DESCRIPTION OF THE FIGURES

As shown in FIGS. 1-4, this invention is directed to a hole forming device 10 useful for creating holes with threads in bone or other appropriate materials. The hole forming device 10 may be formed from an inner shaft 12 and outer sleeve 14 extending distally from a handle 16. The inner shaft 12 may include a pointed tip 18 that may be used to initiate hole formation. Once the hole forming device 10 has been partially inserted into a material, such as, but not limited, bone, the inner shaft 12 may be removed without having to remove the outer sleeve 14. Thus, the inner shaft 12 facilitates insertion of the outer sleeve into the material where the threads on the outer sleeve may engage the bone. The outer sleeve 14 may then be advanced to create threads within the hole. Screws and other appropriate devices may be secured in the threaded hole after the outer sleeve has been removed.

The hole forming device 10 may include a handle 16. The handle 16 may be ergonomically shaped for gripping and ease of use. The handle 16 may be formed from any appropriate material, such as, but not limited to, stainless steel, aluminum, and ABS plastic. The handle 16 may be generally elongated and may have a generally cylindrical outer surface. The handle 16 may include a chamber 20 for receiving the outer sleeve 14. The chamber 20 may extend for a portion of or the entire length of the handle 16 and may include a distal opening 22 and a proximal opening 24. The chamber 20 may be sized to receive the outer sleeve 14.

The outer sleeve 14 may be supported by the handle 16 and the outer sleeve 14 may extend distally from the handle 16. The outer sleeve 14 may extend partially into the handle 16, may extend through the entire length of the handle 16 or may extend completely through the handle 16. The outer sleeve 14 may include a hollow chamber 26 extending through a portion of or through an entire length of the outer sleeve 14 with a proximal opening 28 in the handle 16 and a distal opening 30. The outer sleeve 14 may be formed from any appropriate material, such as, but not limited to, stainless steel. The outer sleeve 14 may be attached to the handle 16 in any appropriate way, including, but not limited to, an interference fit, adhesives, and mechanical connectors. The outer sleeve 14 may include one or more threads 50 extending radially outward from an outer surface 32 of the outer sleeve 14 near the distal end of the outer sleeve 14. The outer sleeve 14 may be a generally elongated hollow sleeve with a cylindrical outer surface 32. The outer sleeve 14 may include a tapered outer surface 32 at the distal end of the outer sleeve 14.

The inner shaft 12 may be positioned within the outer sleeve 14 and may include a pointed tip 18 that extends distally from the distal opening 22 of the outer sleeve 14. When assembled, a proximal end 34 of the inner shaft 12 may be exposed such that the proximal end 34 of the inner shaft 12 can be struck while the inner shaft 12 is positioned in the outer sleeve 14. The inner shaft 12 may be formed from any appropriate material, such as, but not limited to, stainless steel. The inner shaft 12 may be sized to fit within the outer sleeve 14 without unnecessary radial movement and without unnecessary binding. The inner shaft 12 may have an outer diameter that provides a smooth transition to the outer sleeve 14 at a location on the inner shaft 12 that coincides with a location of a distal end of the outer sleeve 14 when the inner shaft 12 is positioned in the outer sleeve 14. In one embodiment, an overall length of the inner shaft 12 may be greater than a combined overall length of an assembled outer sleeve 14 and handle 16.

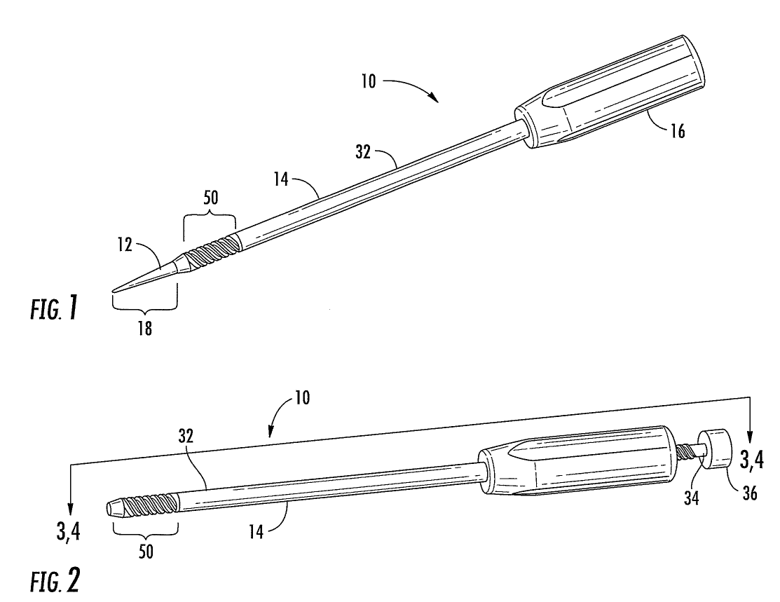

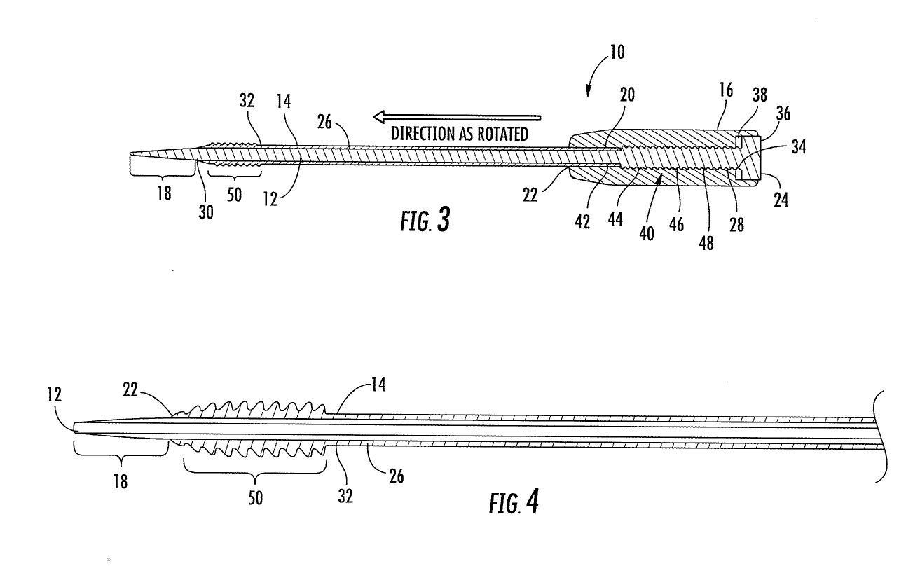

In at least one embodiment, the inner shaft 12 may include a head 36 coupled to the inner shaft 12 at the distal end 34 of the inner shaft 12. The head 36 may be formed to be easily struck by a hammer or other blow delivering device. As shown in FIG. 2, the head 36 may be a generally cylindrical cap attached to the proximal end of the inner shaft 12. The handle 16 may include a head cavity 38 positioned at a proximal end of the handle 16 such that at least a portion of the head 36 resides within the head cavity 38. In one embodiment, the head 36 may fit entirely within the head cavity 38. As shown in FIG. 4, the inner shaft 12 may be formed from a cannula. In another embodiment, as shown in FIG. 3, the inner shaft 12 may be formed from a solid material.

The inner shaft 12 may be releasably attached to the outer sleeve 14 or to the handle 16 or to both using a releasable connector 40. The releasable connector 40 may be any connector 40 that enables the inner shaft 12 to be retracted a sufficient distance such that the pointed tip 18 does not extend distally from the distal opening 22 of the outer sleeve 14. In at least one embodiment, the releasable connector 40 may be formed from one or more threads 44 that extend radially outward from an outer surface 42 of the inner shaft 12 and one or more threads 46 extending radially inward from an inner surface 48 forming the hollow chamber 26 in the outer sleeve 14 such that the one or more threads of the inner shaft 12 and the outer sleeve 14 may interlock, as shown in FIG. 3. As shown in FIG. 3, the threads 44 that extend radially outward from the outer surface 42 of the inner shaft 12 may be positioned near the proximal end of the inner shaft 12, and the threads 46 that extend radially inward from the inner surface 48 of the outer sleeve 14 may be positioned near the proximal end of the handle 16. The threads 44, 46 provide a manner of connection between the inner shaft 12 and the outer sleeve 14. In addition, the threads 44, 46, enable a force to be applied to head 36 directed toward the tip 18 while the handle 16 and outer sleeve 14 are rotated. Application of such a force keeps the tip 18 inside the bone socket while the outer sleeve 16 is free to be rotated, thereby causing the outer sleeve 14 to be pulled with less resistance into the bone socket because of the threads 50.

The hole forming device 10 can be used as a hole forming device and can also be used with currently existing bone sockets such that a currently existing bone socket may be tapped into the socket. The advantage of this usefulness is evident when bone-tendon-bone grafts are utilized in ACL reconstruction. In particular, during ACL reconstruction, the bone graft is slightly undersized from the socket leaving only a small clearance. The inner shaft 12 of the hole forming device 10 assists a user in locating the clearance to assist in inserting the outer sleeve 14 into the hole to start rotating the outer sleeve 14 and cutting threads into the wall forming the hole. Unlike conventional system in which a punch must be removed from a hole and a tap inserted into the hole without guidance, the hole forming device 10 allows a user to tap an entire length of a hole with threads.

During use, the hole forming device 10 may be placed in contact with a material into which a hole is desired to be created. A hammer, or other appropriate force delivering device, may be used to hit the head 36 of the inner shaft 12 contained within the handle 16 and the outer sleeve 14 while the hole forming device 10 is held in place by grasping the handle 16. Once the pointed tip 18 has pierced the material and a distal end of the outer sleeve 14 contacts the material, the inner shaft 12 may be removed entirely from the handle 16 or backed out enough to draw the pointed tip 18 into the outer sleeve 14 while keeping the outer sleeve 14 in place in the material. The handle 16 may then be rotated such that the threads 50 on the outer sleeve 14 cut into the material forming the wall of the hole. The threads 50 on the outer sleeve 14 cause threads to be formed in the wall forming the hole, thereby creating a threaded hole once the outer sleeve 14 is removed from the hole.

The foregoing is provided for purposes of illustrating, explaining, and describing embodiments of this invention. Modifications and adaptations to these embodiments will be apparent to those skilled in the art and may be made without departing from the scope or spirit of this invention.

We claim:

1. A hole forming device, comprising:
a handle;
an outer sleeve supported by the handle and extending distally from the handle, wherein the outer sleeve includes a hollow chamber extending an entire length of the outer sleeve with a proximal opening in the handle and a distal opening;
an inner shaft positioned within the outer sleeve and including a pointed tip that extends distally from the distal opening of the outer sleeve;
wherein the outer sleeve includes at least one thread extending radially outward from an outer surface of the outer sleeve near the distal end of the outer sleeve; and
wherein a proximal end of the inner shaft is exposed such that a proximal end of the inner shaft can be struck while the inner shaft is positioned in the outer sleeve.

2. The hole forming device of claim 1, wherein the inner shaft further comprises a head coupled to the inner shaft at the proximal end.

3. The hole forming device of claim 2, wherein the handle comprises a head cavity positioned at a proximal end of the handle such that at least a portion of the head resides within the head cavity.

4. The hole forming device of claim 1, further comprising a releasable connector that releasably attaches the inner shaft to the outer sleeve.

5. The hole forming device of claim 4, wherein the releasable connector is formed from at least one thread that extends radially outward from an outer surface of the inner shaft and at least one thread extending radially inward from an inner surface forming the hollow chamber in the outer sleeve such that the at least one thread of the inner shaft and the outer sleeve interlock.

6. The hole forming device of claim 5, wherein the at least one thread that extends radially outward from the outer surface of the inner shaft is positioned near the proximal end of the inner shaft, and the at least one thread that extends radially inward from the inner surface of the outer sleeve is positioned near the proximal end of the handle.

7. The hole forming device of claim 1, wherein the outer sleeve is a generally elongated hollow sleeve with a cylindrical outer surface and a tapered outer surface at the distal end of the outer sleeve.

8. The hole forming device of claim 7, wherein the inner shaft has an outer diameter that provides a smooth transition to the outer sleeve at a location on the inner shaft that coincides with a location of a distal end of the outer sleeve when the inner shaft is positioned in the outer sleeve.

9. The hole forming device of claim 1, wherein the handle has a generally cylindrical outer surface.

10. The hole forming device of claim 1, wherein an overall length of the inner shaft is greater than a combined overall length of an assembled outer sleeve and handle.

11. The hole forming device of claim 1, wherein the inner shaft is a cannula.

12. The hole forming device of claim 1, wherein the inner shaft is formed from a solid material.

13. A hole forming device, comprising:
a handle;
an outer sleeve supported by the handle and extending distally from the handle, wherein the outer sleeve includes a hollow chamber extending an entire length of the outer sleeve with a proximal opening in the handle and a distal opening;
an inner shaft positioned within the outer sleeve and including a pointed tip that extends distally from the distal opening of the outer sleeve;
wherein the outer sleeve includes at least one thread extending radially outward from an outer surface of the outer sleeve near the distal end of the outer sleeve;
wherein a proximal end of the inner shaft is exposed such that a proximal end of the inner shaft can be struck while the inner shaft is positioned in the outer sleeve;
a releasable connector that releasably attaches the inner shaft to the outer sleeve; and
wherein the releasable connector is formed from at least one thread that extends radially outward from an outer surface of the inner shaft and at least one thread extending radially inward from an inner surface forming the hollow chamber in the outer sleeve such that the at least one thread of the inner shaft and the outer sleeve interlock.

14. The hole forming device of claim 13, wherein the inner shaft further comprises a head coupled to the inner shaft at the proximal end, and the handle comprises a head cavity positioned at a proximal end of the handle such that at least a portion of the head resides within the head cavity.

15. The hole forming device of claim 13, wherein the at least one thread that extends radially outward from the outer surface of the inner shaft is positioned near the proximal end of the inner shaft, and the at least one thread that extends radially inward from the inner surface of the outer sleeve is positioned near the proximal end of the handle.

16. The hole forming device of claim 13, wherein the outer sleeve is a generally elongated hollow sleeve with a cylindrical outer surface and a tapered outer surface at the distal end of the outer sleeve.

17. The hole forming device of claim 16, wherein the inner shaft has an outer diameter that provides a smooth transition to the outer sleeve at a location on the inner shaft that coincides with a location of a distal end of the outer sleeve when the inner shaft is positioned in the outer sleeve.

18. The hole forming device of claim 13, wherein an overall length of the inner shaft is greater than a combined overall length of an assembled outer sleeve and handle.

19. The hole forming device of claim 13, wherein the inner shaft is a cannula.

20. A hole forming device, comprising:
a handle;
an outer sleeve supported by the handle and extending distally from the handle, wherein the outer sleeve includes a hollow chamber extending an entire length of the outer sleeve with a proximal opening in the handle and a distal opening;
an inner shaft positioned within the outer sleeve and including a pointed tip that extends distally from the distal opening of the outer sleeve;
wherein the outer sleeve includes at least one thread extending radially outward from an outer surface of the outer sleeve near the distal end of the outer sleeve;
wherein a proximal end of the inner shaft is exposed such that a proximal end of the inner shaft can be struck while the inner shaft is positioned in the outer sleeve;
a releasable connector that releasably attaches the inner shaft to the outer sleeve;
wherein the releasable connector is formed from at least one thread that extends radially outward from an outer surface of the inner shaft and at least one thread extending radially inward from an inner surface forming the hollow chamber in the outer sleeve such that the at least one thread of the inner shaft and the outer sleeve interlock;
wherein the at least one thread that extends radially outward from the outer surface of the inner shaft is positioned proximate to the proximal end of the inner shaft, and the at least one thread that extends radially inward from the inner surface of the outer sleeve is positioned proximate to the proximal end of the handle;
wherein the inner shaft further comprises a head coupled to the inner shaft at the proximal end, and the handle comprises a head cavity positioned at a proximal end of the handle such that at least a portion of the head resides within the head cavity; and
wherein an overall length of the inner shaft is greater than a combined overall length of an assembled outer sleeve and handle.
